(19) United States
(12) Patent Application Publication
(10) Pub. No.: US 2010/0145274 A1
(43) Pub. Date: Jun. 10, 2010

(54) LIQUID PHARMACEUTICAL IDENTITY AND QUANTITY MARKING

(76) Inventor: Jan Royce, Stockton, CA (US)

Correspondence Address: ITALIA IP, 3500 WEST OLIVE AVE., SUITE 300, BURBANK, CA, 91505, US

(21) Appl. No.: 12/331,753

(22) Filed: Dec. 10, 2008

(51) Int. Cl. A61M 37/00; A61B 19/00

(52) U.S. Cl. 604/132; 604/131; 128/898

(57) ABSTRACT

Rendering conspicuous liquid medicaments which are conventionally supplied as colorless liquids. A coloring agent may be added to the conventionally colorless liquid. Where different liquid medicaments are supplied, coloring agents of different hues may be added to the respective different liquid medicaments. A syringe for dispensing liquids may be provided with a temperature responsive device which changes in appearance so as to indicate level of liquid medicament loaded thereinto, responsively to temperature of the liquid medicament.

### FIELD OF THE INVENTION

The present invention relates to identifying pharmaceutical medicaments as to identification of the type of medicament and as to quantity occupying a vessel, more particularly by color coding liquid medicaments.

### BACKGROUND OF THE INVENTION

Medicaments may be provided in liquid form or in solid form. When provided in solid form, such as pills, caplets, ampules, tablets, and the like, the medicament may be identified as to its identity and quantity as pills, caplets, and the like are manufactured in standard dosages which may be identified as to quantity and type of the active ingredient. Solid form medicaments therefore present no difficulty to the patient.

However, the situation is different with medicaments in liquid form. Many liquid medicaments are provided in quantities intended to be dispensed little by little, such as in predetermined doses. The final user must measure out a portion of the liquid supply into a spoon, a syringe, or other delivery apparatus. Medicaments to be taken by spoon are easy to dispense. Usually, it is merely necessary to fill the spoon, and then swallow the contents. In this case, it is merely necessary to be sure that the correct medicament is being taken. Normally, this is not a problem. However, it is possible that a patient must take several different liquid medicaments. If each of these is provided as a clear liquid, it is possible to lose track of which specific medicament is being or has been dispensed.

A further problem is when a measuring device such as a syringe is to be used for dispensing. Syringes typically have greater capacity than the amount which is to be taken at any one time. The user must fill the syringe to a prescribed level, then take the medicament.

This may seem simple enough, yet may present difficulties in that it may be difficult to discern when the appropriate amount of a liquid has been loaded into a syringe, especially if a clear liquid is being loaded. This is particularly true if the patient has a problem with vision, even including the usual diminishing of the ability to focus on close objects, which diminishing usually occurs in people over forty years of age. The same problem may occur with medical personnel who may be under time pressure at any given moment.

An example of both problems is seen with insulin. Diabetic insulin is provided to patients as a clear or colorless liquid. A patient may take insulin without any problems pertaining to identification of the insulin. However, some patients must take insulin of different types. Although these different types may be supplied in vials of different sizes, and may have labels on the vials identifying the different types of insulin, there nonetheless remains a potential for the wrong type of insulin to be taken or for the wrong dosage to be taken, or both.

Considering the issue of dosage, insulin may be difficult to measure in a syringe simply because insulin is provided as a clear liquid, such that the level within a syringe is slightly difficult to ascertain visually. This may contribute to improper dosage.

### SUMMARY OF THE INVENTION

The present invention addresses the above problems by providing visual ways to identify different liquid medicaments such as insulin, and to visually assure that the correct dosage is being dispensed within a syringe.

One step to provide visual identification is to color code different types of medicaments by adding a coloring agent to the medicament.

Another way is to modify a syringe to include a temperature sensitive meter which is capable of indicating the level of liquid which fills the syringe.

It is an object of the invention to render medicaments which are normally supplied as colorless liquids more conspicuous.

Another object of the invention is to provide a positive way of ascertaining the level of liquid medicaments within a syringe.

It is an object of the invention to provide improved elements and arrangements thereof by apparatus for the purposes described which is inexpensive, dependable, and fully effective in accomplishing its intended purposes.

These and other objects of the present invention will become readily apparent upon further review of the following specification and drawings.

### BRIEF DESCRIPTION OF THE DRAWINGS

Various objects, features, and attendant advantages of the present invention will become more fully appreciated as the same becomes better understood when considered in conjunction with the accompanying drawings, in which like reference characters designate the same or similar parts throughout the several views, and wherein:

FIG. 1 is a side cross sectional view of a syringe partially loaded with a liquid medicament.

FIG. 2 is a side cross sectional view of a syringe partially loaded with a different liquid medicament.

FIG. 3 is a side view of the syringe of FIG. 1.

### DETAILED DESCRIPTION

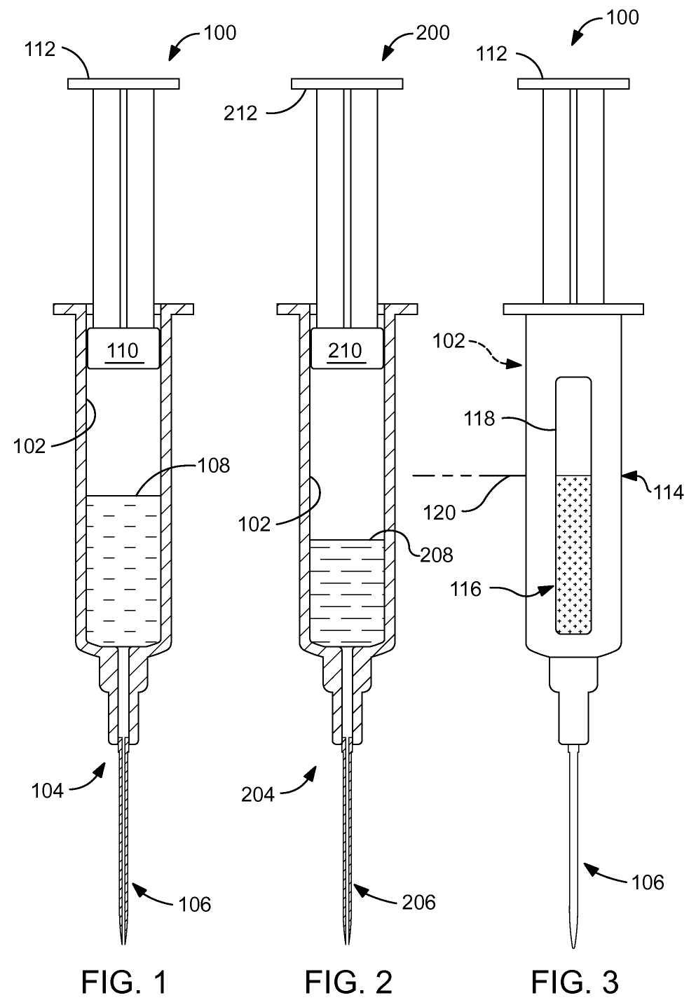

FIG. 1 shows a syringe 100 for injecting liquid medicaments into a person's body, comprising a receptacle 102 having an inlet and outlet orifice 104 which communicates with a hollow needle 106 which is operable for transdermal delivery of liquid medicaments such as a liquid medicament 108 which has been loaded into the receptacle 102.

The syringe 100 may include a plunger 110 disposed to slide axially along the receptacle 102 so as to impose pressure on the liquid medicament 108 when manual pressure is imposed on a finger pad 112 connected to the plunger 110.

The receptacle 102 may be filled or loaded by withdrawing the plunger 110 therefrom, and employing resultant vacuum to draw the liquid medicament 108 into the receptacle 102. Alternatively, the receptacle 102 may be filled or loaded by removing the plunger 110, pouring the liquid medicament 108 into the receptacle 102, and replacing the plunger 110.

The syringe 100 as described thus far may be conventional in structure and function, and need not be set forth in extreme detail.

The liquid medicament 108 may comprise a first composition of liquid diabetic insulin medicament of a first predetermined type to which has been added a coloring agent of a predetermined first hue. Understanding that there are several different types of diabetic insulin, the first predetermined type is merely one of several conventional different types. The coloring agent renders the conventional diabetic liquid insulin visually conspicuous, so that the type of insulin may be identified by correlation with the hue of the coloring agent. It will also be seen that the level of insulin received within the syringe 100 may be more easily discerned compared to traditional colorless or clear liquid insulin medicaments (not shown).

FIG. 2 shows a syringe 200 which may be generally similar structurally and functionally to the syringe 100. The syringe 200 may comprise a receptacle 202 having an inlet and outlet orifice 204 which communicates with a hollow needle 206 which is operable for transdermal delivery of liquid medicaments such as a liquid medicament 208 which has been loaded into the receptacle 202. The syringe 200 may include a plunger 210 disposed to slide axially along the receptacle 202 so as to impose pressure on the liquid medicament 208 when manual pressure is imposed on a finger pad 212 connected to the plunger 210.

The liquid medicament 208 may comprise a second composition of liquid insulin medicament, including a second conventional insulin of a type different from that of the liquid medicament 108, and a coloring agent of a second hue which renders the second conventional insulin visually conspicuous and different from the first composition of liquid insulin medicament, or liquid medicament 108. The type of insulin of the second composition of liquid insulin medicament, or liquid medicament 208, may be identified by comparison with the hue of the coloring agent of the liquid medicament 108. It will also be seen that the level of the second composition of liquid insulin medicament received within the syringe 200 may be more easily discerned compared to traditional colorless liquid insulin medicaments, as well as being visually distinguishable from the liquid medicament 108.

In summary, both the type and the quantity of the liquid medicaments 108 and 208 are readily discernible to ordinary visual inspection due to the two respective coloring agents which have been introduced thereinto.

FIG. 3 shows another way of determining the level of a liquid medicament, such as the liquid medicaments 108 and 208, but also conventional uncolored liquid medicaments. A sensor disposed to sense the level of medicaments received within the receptacle 102 and to indicate the sensed level visually to an observer who is observing the syringe 100 is provided. The sensor is capable of sensing temperature of liquid medicaments received within the receptacle 102, and to indicate the sensed level responsively to sensing the temperature.

The sensor comprises a strip 114 of temperature sensitive material which changes its outer appearance locally responsively to temperature changes occurring along the receptacle 102. This type of material may be of known type which is presently used with beverages, for example. The strip 114 may be locally responsive to temperatures, where temperatures of the liquid medicament may be either higher or lower than that of the receptacle 102. Locally responsiveness of the strip 114 is such that a hue, intensity of hue, or other visually discernible difference occurs in discrete zones where the temperature of the strip 114 is at a particular temperature. This is contrasted for example where the entire strip 114 would display a single color, hue, or other visible effect. For example, a first zone 116 occurs at the lower end (as depicted in FIG. 3) of the strip 114, where the strip 114 is under the influence of the temperature of the liquid medicament contained within the receptacle 102. A second zone 118 of a color, hue, or other visual effect different from that of the first zone 116 is displayed where the strip 114 is at the same temperature of the receptacle 102, assuming of course that the receptacle 102 and the liquid medicament loaded thereinto are at different temperatures.

A line 120 of demarcation is defined at the interface of the zones 116 and 118. This line of demarcation indicates the level of liquid medicament loaded into the receptacle 102 of the syringe 100.

The invention may also be regarded as a method of making a first liquid diabetic insulin medicament readily identifiable by direct observation as to type and quantity. This method may comprise a step of adding a coloring agent to the first liquid diabetic insulin medicament. The method may be expanded to comprise a further step of making a second, different liquid diabetic insulin medicament readily identifiable by direct observation as to type and quantity by adding a coloring agent which is different in hue from that added to the first liquid diabetic insulin medicament.

The present invention is susceptible to modifications and variations which may be introduced thereto without departing from the inventive concepts. For example, although the invention has been described with respect to a singular strip 114 of temperature sensitive material, it would be possible to provide several complementing strips of temperature sensitive material (not shown).

While the present invention has been described in connection with what is considered the most practical and preferred embodiment, it is to be understood that the present invention is not to be limited to the disclosed arrangements, but is intended to cover various arrangements which are included within the spirit and scope of the broadest possible interpretation of the appended claims so as to encompass all modifications and equivalent arrangements which are possible.

I claim:

1. A syringe for injecting liquid medicaments into a person's body, comprising:
a receptacle having an inlet and outlet orifice, and disposed to receive liquid medicaments to be injected;
a plunger disposed to slide axially along the receptacle and to impose pressure on liquid medicaments received within the receptacle;
a hollow needle for transdermal delivery of liquid medicaments which have been loaded into the receptacle; and
a sensor disposed to sense the level of medicaments received within the receptacle and to indicate the sensed level visually to an observer who is observing the syringe.

2. The syringe according to claim 1, wherein the sensor is capable of sensing temperature of liquid medicaments received within the receptacle, and to indicate the sensed level responsively to sensing the temperature.

3. The syringe according to claim 2, wherein the sensor comprises a strip of temperature sensitive material which changes its outer appearance locally responsively to temperature changes occurring along the receptacle, and wherein the strip of temperature sensitive material is disposed along the receptacle such that the level of liquid loaded into the receptacle is indicated responsive to temperature changes.

4. A method of making a first liquid diabetic insulin medicament readily identifiable by direct observation as to type and quantity, comprising the step of adding a coloring agent to the first liquid diabetic insulin medicament.

5. The method of claim 4, comprising the further step of making a second, different liquid diabetic insulin medicament readily identifiable by direct observation as to type and quantity by adding a coloring agent which is different in hue from that added to the first liquid diabetic insulin medicament.

6. A diabetic insulin medicament comprising a first composition of liquid diabetic insulin medicament, comprising conventional diabetic insulin of a predetermined type, and a coloring agent of a first hue which renders the conventional diabetic liquid insulin visually conspicuous, whereby the type of insulin may be identified by correlation with the first hue of the coloring agent, and whereby level of insulin received within a syringe may be more easily discerned compared to traditional colorless liquid insulin medicaments.

7. The diabetic insulin medicament of claim 6, further comprising a second composition of liquid insulin medicament, comprising a second conventional insulin of a type different from that of the first composition of liquid diabetic insulin medicament, and a coloring agent of a second hue which renders the second conventional insulin visually conspicuous and different from the first composition of liquid insulin medicament, whereby the type of insulin of the second composition of liquid insulin medicament may be identified by correlation with the first hue of the coloring agent, and whereby level of the second composition of liquid insulin medicament received within a syringe may be more easily discerned compared to traditional colorless liquid insulin medicaments.
